(19) United States
(12) Patent Application Publication
(10) Pub. No.: US 2009/0280122 A1
(43) Pub. Date: Nov. 12, 2009

(54) USE OF A VIRUS REGIMEN FOR THE TREATMENT OF DISEASES

(75) Inventor: Werner KRAUSE, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: BAYER SCHERING PHARMA AG, BERLIN (DE)

(21) Appl. No.: 12/437,716

(22) Filed: May 8, 2009

Related U.S. Application Data
(60) Provisional application No. 61/052,780, filed on May 13, 2008.

(30) Foreign Application Priority Data
May 9, 2008 (EP) 08075487.2

(51) Int. Cl. A61K 39/395; A61K 35/76

(52) U.S. Cl. 424/137.1; 424/93.6

(57) ABSTRACT

The use of a virus regimen, especially an oncolytic regimen for the production of a medicament for the treatment of a disease, especially cancer is described. The virus regimen is applied after reducing, shutting down or modifying functioning of the immune system in a controlled manner. In a preferred embodiment T-cell depletion or T-cell modification is used for controlling the immune system. The T-cell depletor or T-cell modifier is administered either separately or as part of the virotherapy regimen.

The present invention relates to the use of a virus regimen, especially an oncolytic regimen for the production of a medicament for the treatment of a disease, especially cancer. The virus regimen is applied after reducing, shutting down or modifying functioning of the immune system in a controlled manner. In a preferred embodiment T-cell depletion or T-cell modification is used for controlling the immune system. The T-cell depletor or T-cell modifier is administered either separately or as part of the virotherapy regimen.

The invention involves temporarily shutting down or decreasing the function of the body's immune system either locally or in the whole organism in a controlled way in order to improve the efficacy of virotherapy. In a preferred embodiment the number or the function of T-cells is temporarily reduced. T-cells may also be depleted completely for a limited period of time. The T-cell reducing/depleting/modifying procedure may be performed either before or during virotherapy or can be part of the virotherapy regimen. This procedure is able to effectively improve virotherapy.

Oncolytic virotherapy is a novel, tumor-targeted approach to cancer therapy (A. Stief, Expert Opin. Biol. Ther. (2008) 8(4):463-473). Oncolytic viruses selectively target, infect and kill cancer cells, leaving normal cells intact, thus toxicity to normal tissues should be minimized. Several viruses to date have been identified as having oncolytic potential. These include the DNA viruses: replicating adenovirus, herpes simplex virus, vaccinia virus and myxoma virus; and the RNA viruses: measles virus, vesicular stomatitis virus (VSV), reovirus, Newcastle disease virus, coxsackievirus A21, and others (Russell S J. Cancer Gene Ther 2002; 9: 961-6).

Oncolytic adenoviruses are double-stranded DNA viruses. While non-replicating adenoviruses have been extensively used as gene therapy vectors, replicating adenoviruses have been engineered to be tumor-specific agents. These tumor-targeting properties of adenoviruses have been engineered in three ways: deletion of critical viral genes; insertion of tumor/tissue-specific promoters; and modification of the viral fiber knob used for cell entry. The prototypical tumor-selective replicating adenovirus is ONYX 015, in which the E1B 55K gene was deleted (Heise C, Sampson-Johannes A, Williams A, et al.). ONYX-015 causes tumor-specific cytolysis and antitumoral efficacy that can be augmented by standard chemotherapeutic agents (Nat Med 1997; 3 (6): 639-45).

Measles virus, a member of the paramyxoviridae family, is a negative strand RNA virus. While the wild-type measles virus is a human pathogen, the vaccine strain Edmonston B (MV-Edm) is highly attenuated in normal human cells. Despite this attenuation, MV-Edm is a potent oncolytic virus.

Vesicular stomatitis virus, VSV, is a small, negative strand, RNA virus of the rhabdoviridae family. While it naturally has a wide tissue tropism, it causes a very mild infection in humans, perhaps due to its unique sensitivity to IFN (Rose J K, Whitt M A. In: Fields Virology. Fields B N, Knipe D M, Howley P M, editors. Philadelphia, Lippincott Williams & Wilkins; 2001, p. 1221-43). Phosphorylation of double-stranded RNA-activated protein kinase (PKR) and induction of IFN-responsive genes in normal cells is a critical antiviral response to VSV infection (Stojdl D F, Abraham N, Knowles S, et al. J Virol 2000; 74 (20): 9580-5). Several mutant VSVs that induced IFN production have been described. This resulted in increased protection of mice infected with the mutant VSV compared with the wild type virus thus improving the safety profile of these viruses (Stojdl D F, Lichty B D, Oever B R, et al. Cancer Cell 2003; 4: 263-75). As many cancer cells have defects in their IFN pathways, they have been shown to be supportive of a productive VSV infection and hence selectively killed. VSV has previously been shown to selectively replicate and kill tumors with aberrant p53, ras or myc signalling (Balachandran S, Porosnicu M, Barber G N. J Virol 2001; 75 (7): 3474-9) accounting for up to 90% of cancers.

Reovirus is a double-stranded RNA virus belonging to the reoviridae family (Nibert M L, Schiff L A. In: Fields Virology. Fields B N, Knipe D M, Howley P M, editors. Philadelphia, Lippincott Williams & Wilkins; 2001, p. 1679-720). It causes no known pathology in humans making it an ideal candidate for oncolytic virotherapy. Reovirus was discovered to have oncolytic properties when it replicated preferentially in cancer cells with activated ras pathways ((Coffey M C, Strong J E, Forsyth P A, Lee P W K. Science 1998; 282: 1332-4) and more recently to utilize the ras/ralgef/p38 pathway (Norman K L, Hirasawa K, Yang A-D, et al. Proc Natl Acad Sci USA 2004; 101(30): 11099-104).

A relative newcomer to the field of oncolytic virotherapy, coxsackievirus A21 (CAV21) has been shown to have oncolytic activity in melanoma (Shafren D R, Au G G, Nguyen T, et al. Clin Cancer Res 2004; 10: 53-60) and recently multiple myeloma (Au G G, Lincz L F, Enno A, Shafren D R. Br J Haematol 2007; 137: 133-41). CAV21 is a positive-strand RNA virus and a member of the picornaviridae family (Racaniello V R. Picornaviridae: In: Fields Virology. Knipe D M, Howley P M, editors. Philadelphia, Lippincott, Williams & Wilkins; 2001, p. 685-722). CAV21 is one agent responsible for ‘common-cold’ symptoms in man but has caused no major disease. The tumor-specificity of CAV21 is through its binding to two cellular receptors: intercellular adhesion molecule 1 (ICAM-1) and decay-accelerating factor (DAF), both upregulated in human tumors compared with normal tissues.

Antiviral immune responses may impede delivery and intratumoral spread of oncolytic viruses. Antiviral antibodies neutralize viruses rapidly and irreversibly, raising the concern that a systemically administered oncolytic virus may not persist long enough in the bloodstream to reach the tumor site. The findings by Dingli et al. (Dingli D, Peng K-W, Harvey M E, et al. Biochem Biophys Res Comm 2005; 337: 22-9), suggesting that multiple myeloma patients have significantly fewer anti-measles virus antibodies compared with age matched controls may make this less of a concern for MM patients. Nevertheless, strategies to circumvent the immune response to oncolytic viruses have been proposed. These include utilizing cell carriers as a delivery vehicle for viruses, and inhibiting the interferon response to viral infection. The first response to viral infection of a cell is the activation of early genes including those for the type 1 IFNs.

Type 1 IFNs are potent triggers of the antiviral state through induction of the Janus kinase (Jak)/signal transducers and activators of transcription (STAT) pathway, production of IFN regulatory factors 3 and 7 and ultimately induction of delayed type 1 genes (a second wave of IFN-stimulated genes not induced during initial infection) and genes required for an antiviral state (e.g., PKR and 2′-5′-oligoadenylate synthase; Grandvaux N, tenOever B R, Servant M J, Hiscott J. Curr Opin Infect Dis 2002; 15: 259-67). In order to block one or more steps of the IFN response pathway, viruses encode antagonist molecules such as the P/V/C proteins of paramyxoviruses (Haralambieva I, Iankov I, Hasegawa K, et al. Mol Ther 2007; 15 (3): 588-97). Measles phosphoprotein (P) makes up the basic component of viral RNA polymerase; C and V proteins are non-structural accessory proteins encoded within the P gene. P and V proteins contribute to MV immune circumvention by suppressing STAT1 and STAT2 phosphorylation and inhibiting IFN-induced nuclear translocation of STAT (Haralambieva I, Iankov I, Hasegawa K, et al. Mol Ther 2007; 15 (3): 588-97).

Oncolytic MV (MV-eGFP, an Edmonston strain derivative) induced IFN production in human multiple myeloma and ovarian cancer cells thus inhibiting MV gene expression and virus progeny production in tumor cells. To mitigate this, MV-eGFP was engineered to enhance intratumoral spread by replacing the P (Edmonston) gene with the wild type version (MV-eGFP-Pwt). This virus demonstrated decreased induction of IFN in BJAB lymphoma cells, ARH-77 myeloma cells, and activated peripheral blood mononuclear cells. In vivo, IV MV-eGFP-Pwt showed significantly improved efficacy compared with MV-eGFP in immunocompromised mice bearing human multiple myeloma xenografts. Proteins that counteract innate cellular immune responses are mainly encoded in the P gene, thus there is concern that a recombinant MV expressing a wild type P gene may generate a more toxic agent and compromise patient safety. The strategy to make more potent oncolytic viruses through enhancing the viruses' natural ability to circumvent the innate immune response needs to be balanced with patient safety and warrants further investigation and development.

The Federal Drug Administration (FDA) has not yet approved any human virotherapy product for sale. Current virotherapy is experimental and has not proven very successful in clinical trials.

The question is what factors have kept virotherapy from becoming an effective treatment for disease. Among other factors, the following are of importance
Problems with viral vectors—Viruses, while the carrier of choice in most virotherapy studies, present a variety of potential problems to the patient-toxicity, immune and inflammatory responses, and targeting issues. In addition, there is always the fear that the viral vector, once inside the patient, may recover its ability to cause disease.
Immune response—Anytime a foreign object is introduced into human tissues, the immune system is designed to attack the invader. The risk of stimulating the immune system in a way that reduces virotherapy effectiveness is always a potential risk. Furthermore, the immune system's enhanced response to invaders it has seen before makes it difficult for virotherapy to be repeated in patients.

As described above, there still remains a significant lack of efficacy and risk of complications following virotherapy. The most pressing ones are the immune responses elicited by the viruses, which are identified as foreign by the immune system and the resulting decrease in activity and lack of multiple treatments.

It has now surprisingly been found that shutting down or “dimming” the immune system—for a certain period of time—in a controlled manner in order to prevent the immune system from attacking and inactivating the oncolytic virus will overcome the problems in the art. This can be done by—for example—reducing or eliminating T-cells in the organism or by reducing their functionality. However, any other method of shutting down the immune system or reducing its function may also be utilized. An advantage of the regimen is that the immune system is not damaged but only shut down or reduced in its function and that this effect is reversible. As soon as the oncolytic virus has reached its target and the tumor has started to shrink and lyse, the number/function of T-cells is allowed to return to normal. Additionally, this approach allows for multiple virotherapy treatments during the time in which the immune system is shut down or reduced in its functionality. After discontinuation of treatment, the immune system becomes fully functional again. Depending on the method to shut down or reduce the function of the immune system, it may take some time for the immune system to recuperate its full function, e.g. in the case of T-cell elimination for the normal number of T-cells to reappear. This time not only depends on the specific drug or method used, e.g. for T-cell depletion, but also on the additional use of immune stimulators such as G-CSF or GM-CSF. The re-establishment of a functioning immune system is not restricted to these two examples (G-CSF or GM-CSF). Any other measures known in the art may be used. During the time of treatment and during the time period of recovery of the immune system, the patients are carefully monitored and treated—if necessary—with anti-bacterial drugs in order to prevent or mitigate infections. This prophylaxis is well known to those skilled in the art and constitutes daily life in the treatment of cancer or transplant patients with immune-depressing drugs or T-cell depletors (Semin Hematol. 2004 July; 41(3): 224-33, Leuk Lymphoma 2004 April; 45(4): 711-4).

According to the current invention, patients designated for virotherapy are treated with drugs or methods that are able to shut down or reduce the function of the immune system. In a special embodiment, this is accomplished by killing T-cells or by modifying the function of T-cells. The T-cell depletor/modifier may be part of the virotherapy regimen itself. Drugs of this kind are for example monoclonal antibodies that bind to specific epitopes on T-cells and which effectively kill these cells, such as monoclonal antibodies specific to the CD3 or CD4 antigen. A drug binding to the T3 antigen is muromonab-CD3 (Orthoclone OKT3). Another potential epitope is the CD52 antigen, which is found on B-cells and T-cells. An example for an antibody binding to the CD52 epitope is alemtuzumab (Campath®). However, the invention is not restricted to these types of compounds. Any T-cell depletor/modifier can be used. Also, any epitope on T-cells to which a drug or an antibody can be directed, can be utilized, as can any drug that kills T-cells or reduces their number or functionality. Moreover, any other type of drug that is able to kill T-cells or reduces their number or functioning, i.e. any T-cell depletor or T-cell function modifier, irrespective of their individual mechanisms of action, may be used. Another example for a T-cell depletor is anti-thymocyte globulin, ATG (Thymoglobulin). Thymoglobulin is anti-thymocyte rabbit immunoglobulin that induces immunosuppression as a result of T-cell depletion and immune modulation. Thymoglobulin is made up of a variety of antibodies that recognize key receptors on T-cells and leads to inactivation and killing of the T-cells. Regarding drugs, which modify T-cells, all will be appropriate as long as the result is that the T-cells are either reduced in their number or eliminated or their function is affected. One such exemplary modification is an antibody binding to receptors such as those described above or others, where the binding does not kill T-cells, but modifies its function.

T-cell depletion has been extensively demonstrated for drugs like alemtuzumab or Thymoglobulin. A single dose of alemtuzubmab (Campath®) is able to kill all circulating T-cells. This is illustrated in FIG. 1 (Weinblatt et al. Arth & Rheum 38(11):1589-1594, 1995). As can be seen from FIG. 1, full recovery of T-cells takes 3 months or longer. If the treatment is repeated, T-cell count will remain at low levels or zero during a prolonged period of time. During this period of time multiple virotherapy treatments may be performed without the danger of the immune system eliminating the virus. Alemtuzumab is dosed in CLL three times a week at 30 mg for a total of 4-12 consecutive weeks. The final dose of 30 mg is reached after stepwise increases from 3 mg via 10 mg to 30 mg in the first week. In virotherapy, much smaller doses will be indicated since the tumor load in CLL takes up most of the drug during administration in the first part of the therapy. In multiple sclerosis (MS), where alemtuzumab is also studied, dosing is restricted to five daily doses of 10-30 mg for one week. In MS, the therapy might be repeated after a full year. For virotherapy single doses of 5-10 mg or less might be appropriate.

T-cell depletion after Thymoglobulin is illustrated in FIG. 2 (taken from the Thymoglobulin Prescribing Information). Thymoglobulin is infused in GVHD prevention intravenously over four to six hours. Typical doses are in the range of 1.5-3.75 mg/kg. Infusions continue daily for one to two weeks. The drug remains active, targeting immune cells for days to weeks after treatment. This schedule is routinely adaptable for use in virotherapy.

T-cell depletion for improving virotherapy per this invention is not restricted to the drugs explicitly mentioned herein. Any drug or method that is able to shut down or reduce the function of the immune system may be used. In a special embodiment, drugs or methods that remove, kill or modify T-cells are used. Further examples are described e.g. in Van Oosterhout et al, Blood 2000, 95: 3693-3701. Alternatively, “tetrameric complexes” or ex-vivo T-cell depletion such as immunomagnetic separation (Y. Xiong, The 2005 Annual Meeting, Cincinnati, Ohio) may be used. Other examples include FN18-CRM9, SBA-ER (O′Reilly, Blood 1998; Aversa, JCO 1999), CFE (de Witte, BMT 2000) or leukapheresis using the CliniMACS system. Other physical ex-vivo methods include density gradient fractionation, soybean lectin agglutination+E-rosette depletion, or counterflow centrifugal elutriation. Immunological methods in addition to the ones described above include monoclonal antibodies directed against different receptors on T-cells such as CD6 or CD8. Immunotoxins such as anti-CD5-ricin may also be employed.

As can be seen, the T-cell depletors and modifiers can be used according to the invention in amounts and in administration regimens routinely determinable and analogous to known uses of such agents for other purposes. Preferably, the extent of depletion or loss of function of the T-cells is at least about 50%, 75%, 90%, and also essentially total elimination.

The treatment described above, consisting of T-cell depletion or modification is either adminstered once or until the end of virotherapy depending on the time course of depletion and recovery induced by the drug(s) or procedure(s) selected. Thereafter, the immune system is allowed to recover. Since the system had been shut down in a controlled manner, any T-cells that are newly formed will be fully functional. Recovery of the immune system might be supported by drugs known in the art for this purpose. Examples are G-CSF or GM-CSF. However, any other applicable drugs or measures might as well be utilized.

Another advantage of this invention is that virotherapy can be performed repeatedly on the same patient during the time of immune blockade. Without blocking the immune system, repeated injections of viral treatment that is recognized as “foreign” by the body's immune system will result in a counterattack and—if successful—the virus will be destroyed before being able to reach its target.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosure of the applications, patents and publications, cited herein are incorporated by reference herein.

### BRIEF DESCRIPTION OF THE DRAWINGS

FIGS. 1 and 2 are graphs.

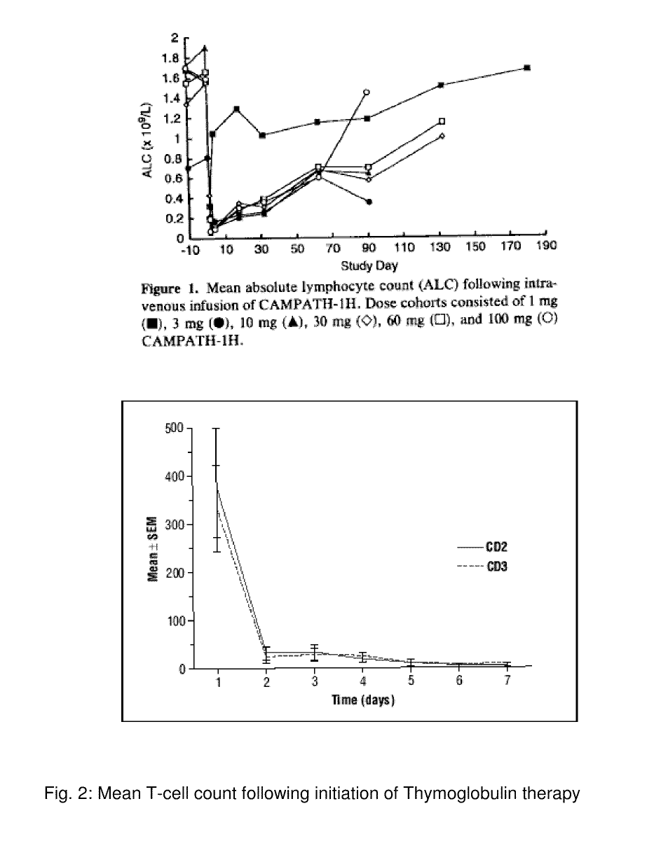

### EXAMPLES

### Example 1

A Phase II study is performed in analogy to the clinical trial NCT00651157 (Viral Therapy of Patients with Malignant Melanoma). In this study the Reovirus Serotype 3-Dearing Strain (Reolysin®) is used for the treatment of melanoma.

### Primary Outcome Measures:

Clinical benefit rate
Tumor response rate

### Secondary Outcome Measures:

Survival time
Time to disease progression
Toxicity as assessed by NCI CTCAE v3.0
Immunologic parameters
Viral replication in metastatic melanoma deposits at 1 week after initiation of treatment
p38 expression in pretreatment tumor specimens
Fludeoxyglucose uptake at baseline and at 4 weeks after initiation of treatment

### Estimated Enrollment: 47

### Objectives:

### Primary

To assess the antitumor effect of wild-type reovirus (Reolysin®) and alemtuzumab, in terms of tumor response rate and clinical benefit rate (i.e., partial response and complete response), in patients with metastatic melanoma.
To assess the toxicity profile of Reolysin® and of alemtuzumab (Campath®) in these patients.

### Secondary

To assess the progression-free survival and overall survival of these patients.
To assess viral replication in metastatic melanoma deposits after intravenous administration of Reolysin® and alemtuzumab.
To assess the impact of pre-existing anti-reoviral immunity (as represented by p38 expression in pretreatment tumor specimens) on the efficacy and toxicity of Reolysin® and alemtuzumab.
To measure the effect of Reolysin® and alemtuzumab on the immune system, in terms of dendritic cell activation, T-cell activation, presence of Treg cells in tumor specimens, and the frequency of T cells, B cells, NK cells, and peptide specific cytotoxic T lymphocytes reactive against melanoma differentiation antigen peptides (gp100, MART-1, and tyrosinase).
To assess the induction of melanoma specific immune response, in terms of the presence of melanoma differentiation antigens (gp100, MART-1, and tyrosinase) in tumor specimens.

Patients receive wild-type reovirus (Reolysin®) IV over 60 minutes on days 1-5. Treatment repeats every 28 days for up to 12 courses in the absence of disease progression or unacceptable toxicity. One day prior to virotherapy, alemtuzumab is administered. A single dose of 5 mg alemtuzumab is either infused intravenously over 2 hours or injected subcutaneously. Prophylaxis of immediate and late adverse reactions is performed as described in the alemtuzumab (Campath®) SmPC for the treatment of CLL patients.

Tumor tissue samples are collected at baseline and at 1 week after initiation of treatment for correlative laboratory studies. Tissue samples are analyzed for p38/MAPK activation status by IHC; reoviral replication in metastatic deposits by electron microscopy; and immunologic parameters by IHC. Blood samples are collected at baseline, at 4 weeks after initiation of treatment, and then every 2 months thereafter. Blood samples are analyzed for immunologic parameters by tetramer and ELISPOT technology and for neutralizing antibodies against reovirus

After completion of study treatment, patients are followed every 6 months for 2 years and then annually for up to 5 years.

### Eligibility

Ages eligible for study: 18 years and older
Genders eligible for study: Both
Accepts healthy volunteers: No

### Disease Characteristics:

Histologically or cytologically confirmed malignant melanoma
   All melanomas, regardless of origin, are allowed
   Metastatic disease
Measurable disease, defined as ≧1 lesion that can be accurately measured in ≧1 dimension (longest diameter to be recorded) as ≧20 mm by conventional techniques or as ≧10 mm by spiral CT scan
Must have ≧1 metastatic lesion that can be safely biopsied
Must have received ≧1 prior treatment for metastatic disease
Not a candidate for curative surgery for metastatic disease
No known brain metastases

### Patient Characteristics:

ECOG performance status 0-2
Life expectancy >12 weeks
Total WBC ≧3,000/mcL
Absolute neutrophil count ≧1,500/mcL
Platelet count ≧100,000/mcL
Hemoglobin ≧9 g/dL
Total bilirubin ≦1.5 times upper limit of normal (ULN)
AST ≦2.5 times ULN
Creatinine ≦1.5 times ULN
Troponin-T normal
LVEF ≧50% by ECHO or MUGA
Not pregnant or nursing
Negative pregnancy test
Fertile patients must use effective contraception
Agrees to provide blood and tissue samples for the mandatory translational research component of the study
Must be able to avoid direct contact with pregnant or nursing women, infants, and immunocompromised individuals during study and for ≧3 weeks following the last dose of study agent
No concurrent uncontrolled illness including, but not limited to, any of the following:
   Ongoing or active infection
   Symptomatic congestive heart failure
   Unstable angina pectoris, cardiac arrhythmia, or myocardial infarction within the past year
   Psychiatric illness/social situation that would preclude study compliance
No known HIV positivity
   Patients with a clinical history suggestive of an immunocompromised status are required to undergo HIV testing

### Prior Concurrent Therapy:

See Disease Characteristics
More than 4 weeks since prior chemotherapy (6 weeks for mitomycin C or nitrosoureas) and recovered
More than 2 weeks since prior radiotherapy, immunotherapy, or treatment with small molecule cell cycle inhibitors
No other concurrent investigational agents
No other concurrent anticancer therapy

It is recommended to perform a Phase I study optimizing the dosing schedule and testing the tolerability of the combination treatment prior to the Phase II trial.

### Example 2

A Phase II study is performed in analogy to the clinical trial NCT00602277 (Viral Therapy in Treating Patients With Ovarian Epithelial Cancer, Primary Peritoneal Cancer, or Fallopian Tube Cancer That Did Not Respond to Platinum Chemotherapy). In this study wild-type reovirus Serotype 3-Dearing Strain (REOLYSIN®) (NSC 729968) is used for the treatment of ovarian cancer.

### Primary Outcome Measures:

Maximum tolerable dose of intraperitoneal (IP) wild-type reovirus when administered with fixed dose IV wild-type reovirus (Phase I)
Proportion of patients demonstrating objective antitumor response (partial response and complete response) as measured by RECIST criteria (Phase II)

### Secondary Outcome Measures:

Association of Ras oncogene and molecular markers with objective response
   Estimated enrollment: 70

### Objectives:

### Primary

To determine the safety and tolerability of intravenous (IV) and intraperitoneal (IP) administration of wild-type reovirus (REOLYSIN®) and of alemtuzumab (Phase I)
To determine the maximum tolerated dose of IP REOLYSIN® when used with a fixed dose of IV REOLYSIN® and of alemtuzumab (Phase I)
To determine the objective response rate (complete response and partial response per RECIST criteria) of treatment with IV and IP REOLYSIN® and alemtuzumab in patients with recurrent, platinum-refractory ovarian epithelial, peritoneal, or fallopian tube carcinoma (Phase II)

### Secondary

To identify viral replication in tumor following IV reovirus.
To identify anti-reovirus antibodies in patients being treated with IV and IP REOLYSIN® therapy and with alemtuzumab.
To identify viral replication in the abdominal washings of patients undergoing IV and IP REOLYSIN® and alemtuzumab therapy.
To correlate response to therapy with Ras oncogene status.
To evaluate double-stranded RNA-activated protein kinase activity in tumors.
To correlate molecular predictors of response to REOLYSIN® and alemtuzumab therapy.

OUTLINE: This is a Phase I, dose-escalation study of intraperitoneal (IP) wild-type reovirus when administered with fixed dose IV wild-type reovirus followed by a Phase II study.
Phase I: Patients receive wild-type reovirus IV over 60 minutes on days 1-5 in course 1, followed by insertion of an IP access port. Beginning in course 2, patients receive wild-type reovirus IV over 60 minutes on days 1-5 and wild-type reovirus IP over 10 minutes on days 1 and 2*. Treatment with IV and IP wild-type reovirus repeats every 28 days in the absence of disease progression or unacceptable toxicity.
Phase II: Patients undergo IP access port insertion before beginning treatment. Patients receive wild-type reovirus IV over 60 minutes on days 1-5 and IP (at the maximum tolerated dose determined in phase I) over 10 minutes on days 1 and 2*. Treatment repeats every 28 days in the absence of disease progression or unacceptable toxicity. NOTE: *Patients receive IP wild-type reovirus on days 2 and 3 in course 3.

One day prior to virotherapy, alemtuzumab is administered. A single dose of 5 mg alemtuzumab is either infused intravenously over 2 hours or injected subcutaneously. Prophylaxis of immediate and late adverse reactions is performed as described in the alemtuzumab (Campath®) SmPC for the treatment of CLL patients.

Prior to each IP wild-type reovirus administration, normal saline is administered through the IP catheter and withdrawn for correlative studies in courses 2 and 3 (Phase I) or courses 1 and 2 (Phase II). Patients also undergo a CT-guided percutaneous tumor biopsy on day 2 of course 3 (Phase I or II). Samples are analyzed by immunohistochemistry, RT-PCR, and electron microscopy for the relevant molecular effects of wild-type reovirus on tumor and normal tissue.

After completion of study treatment, patients are followed for up to 12 weeks

Ages eligible for study: 18 years and older
Genders eligible for study: Female
Accepts healthy volunteers: No

### Disease Characteristics:

Histologically confirmed ovarian epithelial, primary peritoneal, or fallopian tube cancer
Recurrent disease after platinum-based chemotherapy.
   Must have experienced disease persistence during primary platinum-based therapy or recurrence within 12 months after completion of platinum-based chemotherapy (“platinum-refractory” or “platinum-resistant” disease)
      A patient receiving a second course of platinum-based chemotherapy for platinum-sensitive disease who then develops persistence or recurrence within 12 months is considered eligible for this trial
Must have measurable disease by RECIST criteria (Phase II)
Must have received ≧1 prior platinum-based cytotoxic chemotherapy regimen (for primary disease) containing carboplatin, cisplatin, or other organoplatinum compound
   Initial treatment may have included any of the following:
      High-dose therapy
      Consolidation therapy
      Intraperitoneal (IP) therapy
      Extended therapy administered after surgical or nonsurgical assessment
   One additional non-cytotoxic regimen (e.g. monoclonal antibodies, cytokines, small-molecule inhibitors, or hormones) for recurrent or persistent disease allowed
No loculated ascites for which IP distribution of virus is not expected to be feasible
No known brain metastases

### Patient Characteristics:

### Inclusion Criteria:

GOG performance status (PS) 0-2 (Karnofsky PS 60-100%)
Life expectancy >12 weeks
Leukocytes ≧3,000/mcL
Absolute neutrophil count ≧1,500/mcL
Hemoglobin ≧10 g/dL
Platelets ≧1,00,000/mcL
Total bilirubin normal
AST/ALT ≦2.5 times upper limit of normal
Creatinine normal
Ejection fraction ≧50% by echocardiogram or MUGA
Cardiac enzymes normal
Not pregnant or nursing
Fertile patients must use adequate contraception (hormonal or barrier method of birth control or abstinence) prior to study entry and for the duration of study participation
Must be able to avoid direct contact with pregnant or nursing women, infants, or immunocompromised individuals while on study and for ≧3 weeks following the last dose of study agent administration
Cardiac conduction abnormalities (e.g., bundle branch block, heart block) are allowed if their cardiac status has been stable for 6 months before study entry

### Exclusion Criteria:

Patients in whom insertion of an IP catheter is not feasible due to surgical contraindications or abdominal and pelvic adhesions
Known HIV infection or hepatitis B or C
Clinically significant cardiac disease (New York Heart Association class III or IV cardiac disease) including any of the following:
   Pre-existing arrhythmia
   Uncontrolled angina pectoris
   Myocardial infarction 1 year prior to study entry
   Compromised left ventricular ejection fraction ≧grade 2 by MUGA or echocardiogram
Uncontrolled intercurrent illness including, but not limited to, ongoing or active infection, or psychiatric illness/social situations that would limit compliance with study requirements

### Prior Concurrent Therapy:

### Inclusion Criteria:

See Disease Characteristics
At least 4 weeks since most recent cytotoxic chemotherapy (6 weeks for nitrosoureas or mitomycin C)
Recovered from adverse events due to agents administered more than 4 weeks earlier
No prior radiotherapy to the abdomen or pelvis
No other concurrent investigational agents
No investigational or commercial agents or therapies other than those described below may be administered with the intent to treat the patient's malignancy

### Exclusion Criteria:

Chronic oral steroids at an equivalent dose of prednisone 5 mg daily
   Inhaled steroids allowed
Patients on immunosuppressive therapy
Concurrent routine prophylactic use of growth factor (filgrastim [G-CSF] or sargramostim [GM-CSF])

It is recommended to optimize the dose of alemtuzumab in combination with REOLYSIN® in a small pre-Phase I study.

### Example 3

A Phase II study is performed in analogy to the clinical trial NCT00348842 (Newcastle Disease Virus (NDV) for Cancer Patients Resistant to Conventional Anti-Cancer Modalities). In this study the oncolytic strain of Newcastle Disease Virus (MTH-68H) is used for the treatment of cancer.

NDV is a virus that is harmful in chicken, but harmless in man. There are two major sub-strains of NDV, one oncolytic and one non-oncolytic. Oncolytic NDV (MTH-68H) preferentially homes and replicates in cancer cells and therefore administration of NDV intravenously or preferentially intra-tumorally, either by direct injection or by injection into an afferent artery, results in direct lysis of tumor cells. NDV activates apoptotic mechanisms in cancer cells and thus results in natural cell death.

Both oncolytic and non-oncolytic NDV were used clinically in hundreds of patients with different types of cancer worldwide. NDV were proved harmless in man. Clinical studies were done for more than a decade and the efficacy of NDV was documented in pre-clinical animal models as well as in man.
Study Type: Interventional
Study Design: Treatment, Non-Randomized, Open Label, Uncontrolled, Single Group Assignment, Safety/Efficacy Study
Official Title Phase II: Safety and Primary Efficacy of Clinical Application of Newcastle Disease Virus and Alemtuzumab for the Treatment of Patients Resistant to All Conventional Modalities

### Eligibility

Genders eligible for study: Both
Accepts healthy volunteers: No

### Criteria

### Inclusion Criteria:

Patients with the following disease category will be eligible:

Patients with metastatic lung cancer, metastatic GI cancer, metastatic urogenital cancer, skin cancer and soft tissue cancer.
Failure to anti-cancer modalities and evidence of progressive disease despite optimal application of all relevant available anti-cancer modalities.
Consenting patients.
The patient should sign a consent form stating that he/she will make sure to avoid any contact with chicken or any other species of birds.

### Exclusion Criteria:

Not fulfilling any of the above criteria.
Moribund patients or patients with life expectancy <3 months.
Karnofsky performance status <60%.
Pregnant or lactating women.
Active local or systemic infections requiring treatment.
Co-morbidity or life-threatening clinical condition other than the basic cancer.

Dosing of the virus is performed as described in the trial NCT00348842. One day prior to virotherapy, alemtuzumab is administered. A single dose of 5 mg alemtuzumab is either infused intravenously over 2 hours or injected subcutaneously. Prophylaxis of immediate and late adverse reactions is performed as described in the alemtuzumab (Campath®) SmPC for the treatment of CLL patients.

It is recommended to optimize the dose of alemtuzumab in combination with NDV treatment in a small study preceding the above-mentioned trial.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosures of all applications, patents and publications, cited herein and of corresponding European application No. 08075487.2, filed May 9, 2008, are incorporated by reference herein.

1. A method for the treatment of diseases, comprising applying a virus regimen after temporarily shutting down or modifying the functionality of the immune system either locally or in the whole organism.

2. A method according to claim 1, characterized in that the virus regimen is an oncolytic virus regimen.

3. A method according to claim 1, characterized in that the disease is cancer.

4. A method according to claim 1, characterized in that the virus regimen comprises a T-cell depletor or a T-cell modifier that reduces the number and/or functionality of T-cells.

5. A method according to claim 4, characterized in that the virus regimen is applied after depleting the T-cells or modifying their functionality.

6. A method according to claim 5, characterized in that the T-cell depletion or modification is performed ex vivo.

7. A method according to claim 4, characterized in that the T-cell depletor or modifier is applied independently of the virus regimen.

8. A method according to claim 4, characterized in that the T-cell depletor or modifier is part of the virus regimen.

9. A method according to claim 4, characterized in that a monoclonal antibody which is directed against CD3 is applied.

10. A method according to claim 4, characterized in that a monoclonal antibody which is directed against CD4 is applied.

11. A method according to claim 4, characterized in that a monoclonal antibody which is directed against CD52 is applied.

12. A method according to claim 4, characterized in that muromonab-CD3 is applied.

13. A method according to claim 4, characterized in that alemtuzumab is applied.

14. A method according to claim 4, characterized in that an anti-thymocyte globulin is applied.

15. A method according to claim 4, characterized in that the T-cell suicide gene transduction (Tk-gene) is applied.

16. A method according to claim 4, characterized in that the T-cell depletor or T-cell modifier is applied prior to the virus regimen use.

17. A method according to claim 4, characterized in that the T-cell depletor or T-cell modifier is applied or acts until one or multiple rounds of virus regimen have successfully been applied.

18. A method according to claim 1, characterized in that the T-cell depletion/modification is accompanied or followed by a treatment for strengthening of the immune system.

19. A method according to claim 1, characterized in that the T-cell depletor or modifier is used in combination with or is applied followed by a G-CSF or GM-CSF treatment.

20. A method according to claim 1, characterized in that the T-cell depletor essentially eliminates T-cells.

21. A method according to claim 4, characterized in that a T-cell modulator is administered.

22. A method according to claim 1, characterized in that the T-cell modulator essentially silences T-cells.

23. A method according to claim 1, characterized in that the extent of T-cell depletion is at least 50%.

24. A method according to claim 1,
characterized in that the extent of T-cell function loss is at least 50%.
